(19) United States
(12) Patent Application Publication
(10) Pub. No.: US 2006/0198889 A1
(43) Pub. Date: Sep. 7, 2006

(54) Roflumilast and integrin inhibitor combination and treatement method

(76) Inventors: Harpreet K. Sandhu, West Orange, NJ (US); David J. Valacer, San Francisco, CA (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/365,569

(22) Filed: Mar. 1, 2006

Related U.S. Application Data
(60) Provisional application No. 60/658,719, filed on Mar. 4, 2005.

(51) Int. Cl. A61K 38/04; A61K 9/26

(52) U.S. Cl. 424/470; 514/19

(57) ABSTRACT

The present invention provides novel solid pharmaceutical dosage forms for oral administration comprising a therapeutically active amount of roflumilast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients. These novel solid pharmaceutical dosage forms are useful in the treatment or control of asthma. The present invention also provides a method for treating asthma employing the solid pharmaceutical dosage forms and a method for preparing the pharmaceutical dosage forms.

### PRIORITY TO RELATED APPLICATIONS

This application claims the benefit of U.S. Provisional Application No. 60/658,719, filed Mar. 4, 2005, which is hereby incorporated by reference in its entirety.

### FIELD OF THE INVENTION

The present invention provides novel solid pharmaceutical dosage forms for oral administration comprising a therapeutically active amount of roflumilast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients. These novel solid pharmaceutical dosage forms are useful in the treatment or control of asthma and allergic rhinitis. The present invention also provides a method for treating asthma employing the solid pharmaceutical dosage forms and a method for preparing the pharmaceutical dosage forms.

### BACKGROUND OF THE INVENTION

Asthma

Asthma is a chronic inflammatory disorder of the airways characterized by a reduction in lung function and airway hyper-responsiveness (AHR). The airway abnormalities in asthmatics are characterized by constriction, which is the tightening of the smooth muscles surrounding the airways, and inflammation, which is the swelling and irritation of the airways and mucus plugging of small airways caused by mucus hypersecretion. Constriction, plugging and mucosal inflammation contribute to obstruction of airflow, which results in symptoms such as wheezing, coughing, chest tightness, and shortness of breath.

Airway inflammation is a hallmark of asthma. Several studies have documented an association between the numbers of eosinophils and activated lymphocytes in the airways and clinical indices of disease severity. Eosinophils are thought to be important effectors involved in bronchial mucosal damage by the release of cationic proteins, reactive oxygen species, and proinflammatory and profibrotic mediators. Much emphasis has been placed on CD4+ T helper type 2 (Th2) cells as central promulgators of this inflammatory process. These Th2 lymphocytes are believed to orchestrate the events leading to the development of allergic airway responses mainly through the production of Th2-type mediators, which in turn promote the eosinophil-rich infiltrate that distinguishes asthmatic airway inflammation. Although there are available therapies focused on reducing this chronic inflammatory process in asthma, no currently available treatment has been shown to eliminate all features of the disease as a singularly effective treatment. Significant unmet medical needs remain in asthma management for patients with moderate to severe disease.

Early treatment for asthma is focused on relief of the smooth muscle contraction that leads to bronchoconstriction. A variety of medications have been used to provide quick relief and/or prevent bronchoconstriction and the resultant symptoms, e.g., wheeze, cough, exercise intolerance, and/or shortness of breath. Widely used relievers of bronchoconstriction include inhaled short-acting beta-adrenoceptor agonists such as salbutamol and albuterol, their long acting inhaled counterparts, salmeterol and fomoterol and orally administered long acting roflumilast. In addition to these inhaled beta-adrenoceptor agonists, there are controller medications that reduce airway inflammation through daily administration on a long-term basis. Inhaled corticosteroids (ICS) are the most potent and effective anti-inflammatory medications and are the first line of therapy for asthma patients. After a decade of widespread use of inhaled corticosteroids therapy, several respiratory health organizations have produced survey data, which concludes that a majority of moderate to severe asthma patients do not enjoy complete and optimal control of their symptoms as defined by the widely accepted GINA/NIH (Global Initiative For Asthma/National Institutes of Health) guideline-based treatment goals. Even with higher doses of inhaled corticosteroids most patients continue to require beta agonist bronchodilator therapy and current U.S. and international asthma treatment guideline recommend that patients with more. than mild asthma be treated daily with both anti-inflammatory and long acting bronchodilator therapy. Many more severe asthmatics are actually treated with high dose inhaled corticosteroids as well as one or more additional anti-inflammatory drug daily in order to attain guideline directed levels of disease control and improved quality of life.

However, the deleterious side effects of these higher doses of inhaled corticosteroids given long-term often outweigh the clinical benefits for some patients. In addition, many studies have shown that patient adherence to long term daily treatment with two or more medications is very poor. In general, studies have shown highest compliance in chronic disease therapy can be achieved with once daily oral administration dosing regimens. For this reason, the search for better, orally administered, complementary “controller” treatments that can both spare asthma patient exposures to higher doses of Inhaled corticosteroids as well as reduce their number of daily medications and frequency of dosing has been widely advocated to provide better asthma control and prevent progression of the disease.

Role of Eosinophils In Asthma

The role of eosinophils in asthma is described in detail in Busse, W. W. et al., N. Engl. J. Med. 2001; 344-350, which disclosure is incorporated herein by reference. Inhaled antigens activate mast cells and Th2 cells in the airway, which in turn induce the production of mediators of inflammation such as histamine, leukotrienes and chemokines, including interleukin-4 and interleukin-5. Interleukin-5 in the bone marrow causes terminal differentiation of eosinophils. Circulating eosinophils enter the area of allergic inflammation and begin migrating to the lung by rolling, through interactions with selectins, and eventually adhering to the endothelium through the binding of integrins to members of the immunoglobulin superfamily of adhesion proteins: vascular-cell adhesion molecule 1 (VCAM-1) and intercellular adhesion molecule 1 (ICAM-1). As the eosinophils enter the matrix of the airway through the influence of various chemokines and cytokines (such as MCP-1, monocyte chemotactic protein, and MIP-1 (macrophage inflammatory protein), their survival is prolonged by interleukin-5 and granulocyte-macrophage colony-stimulating factor (GM-CSF). On activation, the eosinophil releases inflammatory mediators such as leukotrienes and granule proteins to injure airway tissues. In addition, eosinophils can generate granulocyte-macrophage colony-stimulating factor to prolong and potentiate their survival.

The presence of activated CD4 Th2 cells is also a hallmark feature of asthma in particular of chromic asthma. The persistence of Th2 cells may be the result of an increased recruitment and a prolonged survival in the airway tissue interstium (Cohn L, Elias J A, Chupp G L. Annual Review of Immunology. 2004. 22 (1): 789-815). As with eosinophils, Th2 cells enter the airways from the vascular through interaction of adhesion molecules with the vascular endothelium. Once in the tissue, these cells encounter antigen presenting cells, such as dendritic cells, where they proliferate. This costimulatory response as well as the resistance to apoptosis may be mediated by alpha4-VCAM-1 interactions.

Early and Late Phase Reactions to Allergens

In controlled inhaled allergen challenge experiments, sensitized asthmatic patients develop an early-phase allergic response (EAR) that begins within minutes of allergen exposure and most often resolves spontaneously after 30 to 60 minutes. This early-phase allergic response results primarily from the release of preformed pro-inflammatory mediators such as histamine as well as the de novo generation of leukotrienes C₄, D₄, and E₄ by bronchial mast cells. These mediators induce smooth muscle contraction, mucus secretion, and vasodilatation. Inflammatory mediators also induce microvascular leakage of plasma proteins, causing edematous swelling of the airway walls and a narrowing of the airway lumen.

This early-phase allergic response is usually followed by a second phase of airflow obstruction, termed the late-phase allergic response (LAR), which occurs 6 to 10 hours later. The late-phase allergic response develops as a result of cytokines and chemokines generated by resident cells of the lung (mast cells, macrophages, and epithelial cells) and recruited inflammatory cells (T lymphocytes and eosinophils). The T lymphocytes involved in this process are of the Th2 type and are found in a wide variety of hypersensitivity reactions including allergic rhinitis as well as asthma. Th2 cells produce interleukins, which have pronounced effects on inflammatory cells, particularly eosinophils. Circulating eosinophils migrate into the airway. Upon activation, eosinophils release inflammatory mediators such as leukotrienes, and granule proteins such as major basic protein which injure airway tissues. Features of the late-phase allergic response include bronchospasm, escalating inflammation, mucous hypersecretion and airway wall edema. Swelling of the airway wall also leads to a loss of elasticity, further contributing to chronic airflow limitation. An additional consequence of the late-phase allergic response is an increase in airway hyper-responsiveness, which reinforces and perpetuates the asthmatic response.

The Integrins

The integrins constitute a large class of heterodimeric, cell surface molecules consisting of α and β chains, each of which has a large extracellular domain and a short cytoplasmic tail. There are at least 14 different α chains and 8 β chains known, which combine in a restricted manner depending on cell type to give approximately 23 members of the integrin family, each of which binds specific peptide ligands. Integrins mediate a variety of cell functions including adhesion, migration, activation and survival. Lymphocytes and leukocytes with the exception of neutrophils constitutively express the integrin VLA-4 (α₄, very late activating antigen4, CD49d/CD-29) and are capable of expressing the closely related integrin, α₄β₇.

The α₄β₁ and α₄β₇ integrins mediate cell-cell adhesion to the immunoglobulin superfamily member, vascular cell adhesion molecule-1 (VCAM-1), and cell-matrix adhesion to fibronectin. In addition, α₄β₇ also binds mucosal addressin cell adhesion molecule-1 (MadCAM-1). VCAM-1 regulates leukocyte migration from the blood into tissues. VCAM-1 expression is induced on endothelial cells during inflammatory responses such as that seen in asthma.

In asthma, there is increased expression of α₄β₁ and α₄β₇ integrins on all mononuclear leukocytes (including Th2 cells), eosinophils, basophils, and mast cells. The selective and increased expression of the a4 integrins only on those cells involved in the inflammatory cascade in asthma would suggest that it is possible to target the underlying disease process without compromising normal host-defense responses.

In vivo studies with monoclonal antibodies (MoAbs) to the α₄ chain of α₄β₁ and α₄β₇ in several animal models of asthma demonstrate that α₄ integrins play a key role in eosinophil and T cell recruitment, activation, and survival leading to a significant reduction of airway inflammation. Furthermore, antibodies directed against VLA-4 block eosinophil accumulation, hyper-reactivity, and inflammation in mouse, rat and guinea pig models of allergic asthma. More recently the peptide VLA4 antagonist, Bio1211, was shown to block late phase airway response as well as to attenuate carbacol induced airway hyper-responsiveness in a sheep model of allergic asthma. Lastly, VCAM-deficient mice show no signs of airway inflammation.

R411

R411 (N-(2-Chloro-6-methylbenzoyl)-4-[(2,6-dichlorobenzoyl)amino]-L-phenylalanine-2-(diethylamino)ethyl ester) is an ester pro-drug of the active moiety, N-(2-chloro-6-methylbenzoyl)-4-[(2,6-dichlorobenzoyl)amino]-L-phenylalanine. R411 has the following chemical structure:

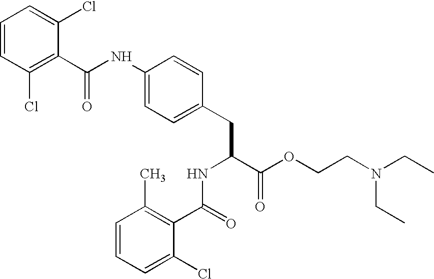

R411 inhibits the binding of α₄/β₁ to vascular cell adhesion molecule (VCAM-1) and α₄/β₇ to MadCAM-1 by binding to R411 is disclosed in U.S. Pat. No. 6,229,011, which disclosure is incorporated by reference herein.

R411 will only modulate immune responses mediated by α₄-integrins and, therefore in asthma, selectively target only those inflammatory cells involved in the pathogenesis of the disease: Th2 cells, eosinophils, and mast cells. The expression of α₄-integrins on these cells is increased in asthma mediating their recruitment, activation, retention, and survival in the airways. The alpha4 integrins appear not to be involved in cellular immunity and other humoral host defense responses. Therefore R411 would be expected to selectively target the inflammatory response in asthma without compromising normal host-defense.

R411 binds with high affinity and slow dissociation from the activated α₄ ligand. In contrast, in vitro binding affinity is lower and dissociation is more rapid when the receptor is not activated. While Bio1211 is specific for α₄/β₁ integrin, R411 is effective against both α₄/β₁ and α₄/β₇ integrins.

R411 can attenuate airway hyper-responsiveness; reduce edema; reduce smooth muscle hypertrophy/mucus gland hyperplasia; block trafficking of leukocytes to airways; increase peripheral blood lymphocytes and eosinophils; modulate Th2 cytokine production; block costimulatory signals for T cells and eosinophils; and inhibit eosinophil survival. In our experimental studies, R411 was observed to block the migration of key inflammatory cells from the blood into the lungs.

Many α-integrin inhibitors having various inhibitory selectivity patterns have been disclosed; see e.g.: U.S. Pat. Nos. 6,380,387; 6,388,084; 6,420,600; 6,423,728; 6,455,550; and 6,734,311.

Roflumilast

Roflumilast (Daxas®) is an oral anti-inflammatory, selective phosphodiesterase type-4 (PDE4) inhibitor for the once-daily treatment of chronic obstructive pulmonary disease (COPD) and asthma. PDE4 inhibitors are useful as bronchial therapeutics for the treatment of airway obstructions because of their dilating action, respiratory drive increasing action, and anti-inflammatory action. The recommended dosage of roflumilast is 0.25-0.5 mg. Roflumilast is 3-cyclopropylmethoxy-N-(3,5-dichloropyridyl)-4-difluoromethoxy)-benzamide (3-cyclopropylmethoxy-4-difluoromethoxy-N-(3,5-dichloropyrid-4yl)benzamide) and has the following chemical structure:

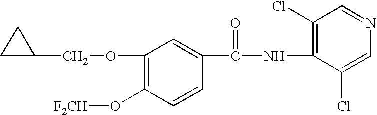

The major metabolite of roflumilast is roflumilast N-oxide which is also pharmacologically active. Roflumilast N-oxide is 3-cyclopropylmethoxy-4-difluoromethoxy-N-(3,5-dichloro-1-oxypyrid-4yl)benzamide.

Roflumilast is disclosed in U.S. Pat. No. 5,712,298, which disclosure is incorporated by reference herein.

United States patent application no. US2002/0052312 discloses a method for treating chronic obstructive pulmonary disease which comprises administering orally a muscarinic M3 receptor antagonist in combination with a therapeutic agent selected from the group consisting of P2-agonist, antitussive, corticosteroid, decongestant, histamine H1 antagonist (antihistamine), dopamine antagonist, leukotriene antagonist, 5-lipooxygenase inhibitor, phosphodiesterase IV inhibitor, VLA4 antagonist, and theophylline.

WO 2004/091596 discloses a method of treating asthma, COPD, allergic rhinitis, and infectious rhinitis by administering a pharmaceutical agent of formulae IV and a second pharmaceutical agent selected from adenosine A2a receptor agonists, D2-dopamine receptor agonists, PDE inhibitors, corticosteroids, norepinephrine reuptake inhibitors, and 4-hydroxy-7-[2-[2-[3-[2-phenylethoxy]-propylsulphonyllethylamino]ethyl]-1,3-benzothiazol-2(3H)-one. WO 2004/084897 dislcoses the administration of roflumilast oral or untravenously and an anticholinergic agent selected from an ipratropium, oxitropium or tiotropium salt for the treatment of respiratory diseases. WO 2004/084896 discloses the administration of roflumilast and an anticholinergic agent selected from an ipratropium, oxitropium or tiotropium salt for the treatment of respiratory diseases. WO 2004/084894 discloses the administration of roflumilast and revatropate for the treatment of respiratory diseases. WO 2003/011274) discloses treating pulmonary diseases by administering a PDE4 inhibitor in combination with an anticholinergic agent. WO 02/096463 discloses an inhalation combination of a PDE4 inhibitor and an anticholinergic agent for the treatment of an obstructive airway disease, with the proviso that the anticholinergic agent is not a tiotropium salt. WO 02/096423 discloses a combination of therapeutic agents useful in the treatment of obstructive airways comprising (I) a PDE4 inhibitor administered by inhalation and (II) an anti-cholinergic agent comprising tiotropium and derivatives thereof. WO 02/069945 discloses compositions comprising anticholinergics and PDE4 inhibitors in the therapy of respiratory tract diseases.

### BRIEF DESCRIPTION OF THE FIGURES

FIG. 1 is a graph illustrating the additive effect of R411 on moderate dose inhaled corticosteroids in large airway flow rates as measured by FEV₁.

FIG. 2 is a graph illustrating the additive effect of R411 on moderate dose inhaled corticosteroids in large airway flow rates as measured by FEF25-75.

FIG. 3 is a graph illustrating the effect of R411 on small airway flow rates as measured by FEF25-75 when administered as monotherapy to asthmatic patients.

FIG. 4 is a bar graph showing that the oral administration of R411 attenuates airway inflammation in the atopic primate.

### SUMMARY OF THE INVENTION

The present invention provides a solid pharmaceutical dosage form for oral administration comprising a therapeutically active amount of roflumilast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, and one or more pharmaceutically acceptable excipients.

The present invention also provides a method for treating asthma comprising administering to a subject, in need thereof, a solid pharmaceutical dosage form for oral administration comprising a therapeutically active amount of roflumilast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

The present invention further provides a method for preparing a solid pharmaceutical dosage form for oral administration comprising admixing a therapeutically active amount of roflumilast, or a pharmaceutically acceptable salt thereof, a therapeutically effective amount of N-(2-chloro-6-methylbenzoyl)4-[(2,6-dichlorobenzoyl)amino]-L-phenylalanine-2-(diethylamino)ethyl ester, or a pharmaceutically acceptable thereof, and one or more pharmaceutically acceptable excipients.

### DETAILED DESCRIPTION OF THE INVENTION

The present invention provides solid pharmaceutical dosage forms for oral administration comprising a therapeutically active amount of roflumilast, or a pharmaceutically acceptable salt thereof, a therapeutically effective amount of R411, or a pharmaceutically acceptable thereof, and one or more pharmaceutically acceptable excipients. In a preferred embodiment, the dosage form comprises a combination of roflumilast and R411 admixed together with pharmaceutical excipients or the active ingredients may be pre-formulated as individual compositions and then mixed to form a unit dose containing a therapeutic amount of each compound. The first composition comprises a therapeutically active amount of roflumilast, or a pharmaceutically acceptable salt thereof, formulated with one or more pharmaceutically acceptable excipients. The second composition comprises a therapeutically effective amount of R411, or a pharmaceutically acceptable salt thereof, formulated with one or more pharmaceutically acceptable excipients. Other preferred solid dosage forms are those in which the separately pre-formulated compositions are combined in a dosage form having separate discrete regions for the two pre-formulated ingredients such as by discrete layers, encapsulations, and the like. Examples of such dosage forms include, but are not limited to, a compressed tablet, a bilayer tablet, a sandwich tablet, a tablet having coated microbeads, or a film coated tablet.

These oral dosage forms are useful in the treatment or control of asthma and allergic rhinitis.

The pharmaceutical dosage forms of the present invention provide two compounds for treating asthma that operate by complementary mechanisms of action. Roflumilast is a selective phosphodiesterase type-4 (PDE4) inhibitor which is a bronchial dilator and exhibits respiratory drive increasing action and anti-inflammatory action. R411 inhibits eosinophil and Th2 cell excitation and survival, and inhibits eosinophil migration from blood to pulmonary tissues. The combination of the two compounds in the pharmaceutical dosage forms therefore provides a therapeutic treatment that has the combined effect of providing dilating action, respiratory drive increasing action, the inhibition of the release of inflammatory mediators and the reduction of eosinophil egress into pulmonary tissues thereby providing an early onset of bronchodilation as well as sustained anti-inflammatory effects. Hence administration of the pharmaceutical dosage forms of the present invention provides a means of intensifying asthma therapy while supporting good patient compliance.

As used herein, the following terms have the given meanings:

“Roflumilast” refers to roflumilast, pharmaceutically acceptable salts thereof, to racemic mixtures, and to pure enantiomers. “Roflumilast” also refers to the major metabolite of roflumilast, roflumilast N-oxide, which is also pharmacologically active.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., H. Ansel et. al., Pharmaceutical Dosage Forms and Drug Delivery Systems (6^{th} Ed. 1995) at pp. 196 and 1456-1457.

“Prodrug” refers to compounds, which undergo biotransformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

“R411” refers to N-(2-chloro-6-methylbenzoyl)-4-[(2,6-dichlorobenzoyl)amino]-L-phenylalanine-2-(diethylamino)ethyl ester, and pharmaceutically acceptable salts thereof.

“Therapeutically effective amount” means an amount of at least one compound of the invention, or a pharmaceutically acceptable salt thereof, which is effective to prevent, alleviate or ameliorate symptoms of disease or prolong the survival of the subject being treated. Determination of a therapeutically effective amount is within the skill in the art.

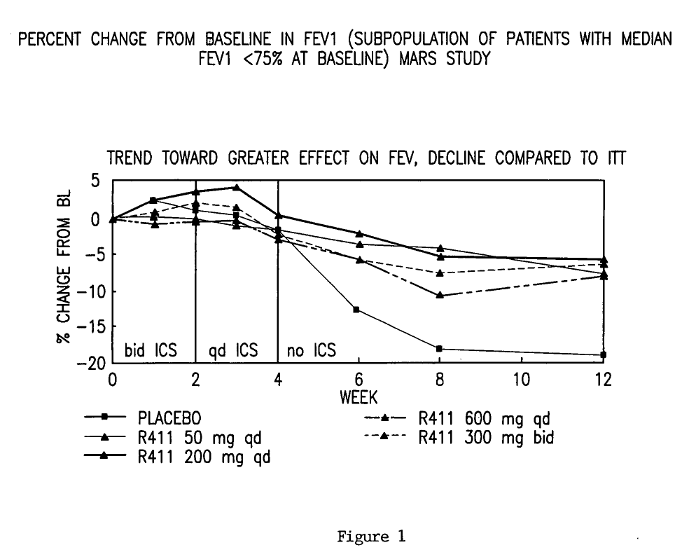

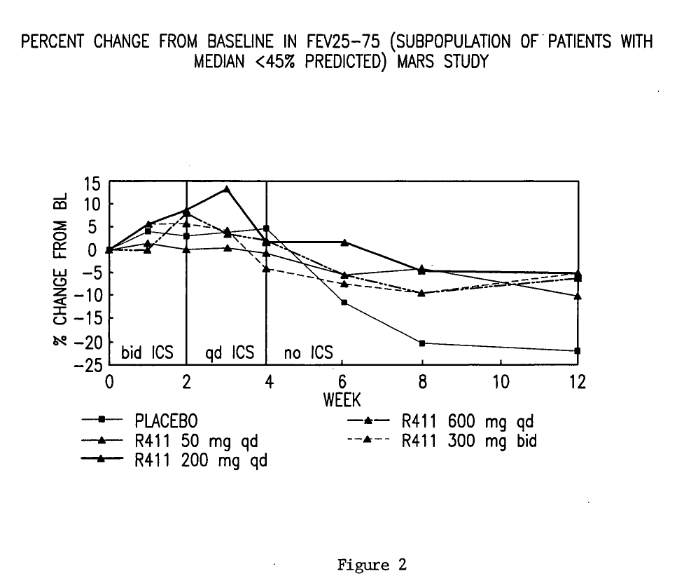

As set out above, a second component in the solid pharmaceutical dosage form comprises a therapeutically effective amount of R411 (N-(2-chloro-6-methylbenzoyl)-4-[(2,6-dichlorobenzoyl)amino]-L-phenylalanine-2-(diethylamino)ethyl ester), or a pharmaceutically acceptable salt thereof. In Phase II studies, R411 demonstrated an additive effect to moderate dose inhaled corticosteroids in large airway flow rates as measured by FEV, (FIG. 1) and small airway flow rates measured by FEF25-75 (FIG. 2) in a subpopulation of patients with not well controlled asthma. The MARS study illustrated in FIG. 2 was designed to evaluate the safety and efficacy of R41 1 over a 12 week treatment period in 350 persistent asthmatics being treated with a stable dose of low to medium inhaled corticosteroids and inhaled short acting β2-agonist. Patients were randomized to one of five cohorts: 50, 200, 600 mg once daily (QD) or 300 mg twice daily (BID) R41 1, or placebo (n=70/group). After a 2-week placebo run-in period and subsequent 2-week add-on period (R411 or placebo), morning inhaled corticosteroids were removed. Two weeks later, evening inhaled corticosteroids were removed, and patients remained in the treatment period for an additional 8 weeks. The primary endpoint in the study was the percentage change in FEV1 from baseline, and secondary endpoint included PEFR, asthma exacerbations, β2-agonist use, asthma control questionnaire, asthma symptom scores, nocturnal awakenings, FEF25-75 and rate of asthma treatment failures.

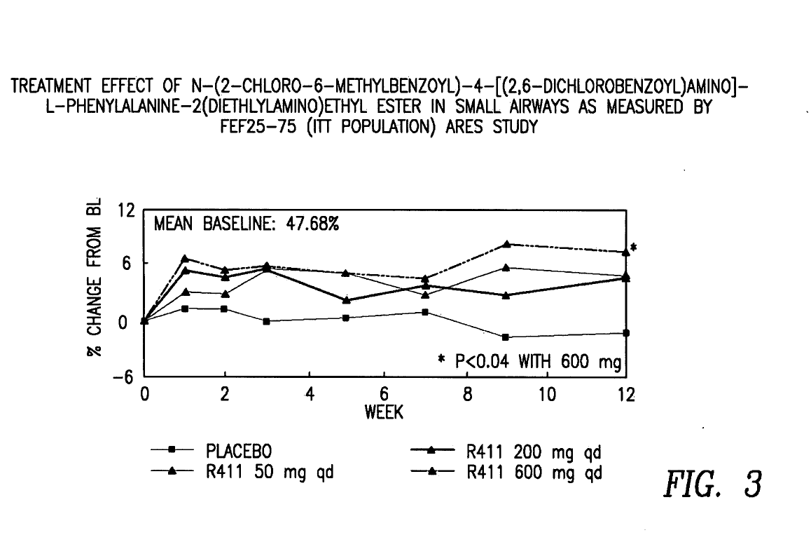

A significant effect on small airway flow rates as measured by FEF25-75 was seen with R411 even when administered as monotherapy to asthmatic patients (FIG. 3). The ARES study illustrated in FIG. 3 was designed to evaluate the safety and efficacy of monotherapy R411 over a 12 week treatment period in 480 mild/moderate asthmatics not treated with inhaled corticosteroids. Patients were randomized to one of four cohorts: 50, 200, 600 mg QD R411, or placebo (n=120/group). The primary endpoint in the study was change in FEV1 from baseline, and secondary endpoints included PEFR, asthma exacerbations, β2-agonist use, asthma control questionnaire, asthma symptom scores, and nocturnal awakenings. Small airway inflammation represents a clinically significant component of moderate to severe asthma that is not adequately controlled by currently available inhaled corticosteroids therapies. Therefore, R411 represents a novel opportunity to address an important unmet need in control of asthmatic airway inflammation.

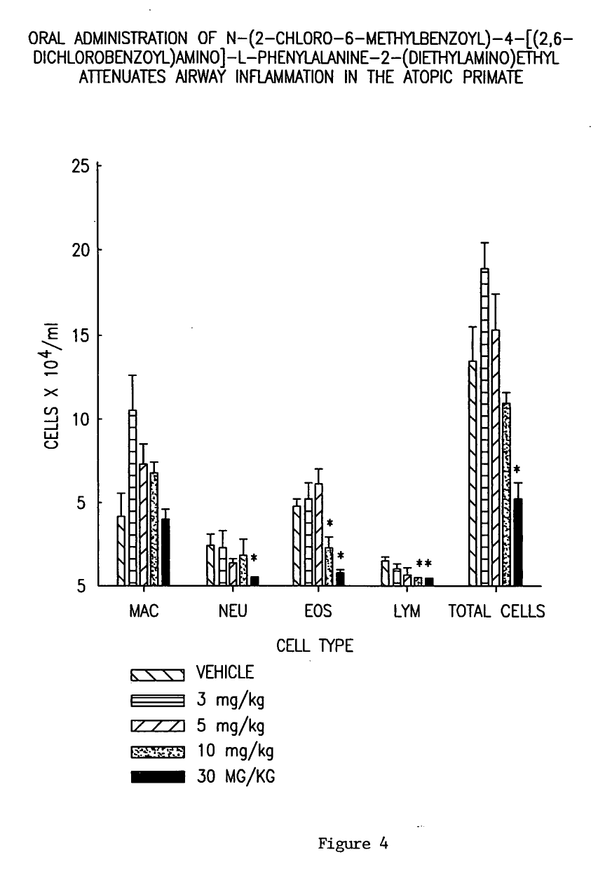

FIG. 4 is a bar graph showing that the oral administration of R411 attenuates airway inflammation in the atopic primate.

Improvement on Asthma and Allergy Symptoms

R411 also has positive effects on symptoms of asthma. ARES study evaluated the safety and efficacy of monotherapy with R411 over a 12-week treatment period in 479 mild/moderate asthmatics not treated with inhaled corticosteroids. Patients were randomized to one of four cohorts: 50, 200, 600 mg once daily R411, or placebo. Statistically significant improvements with R441 were achieved in reducing rescue albuterol use, decrease in daytime asthma and nocturnal symptom score. Improvement in Asthma Control Questionnaire Scores and Asthma Quality-of-Life were also observed when compared to placebo. Although the study was not powered to detect significant differences in asthma exacerbations, a 26% reduction was observed with the two highest doses of 200 and 600 mg. The results are set out in the Table below.

### Secondary Efficacy Endpoints in the ITT Population When R411 is Administered as Monotherapy (ARES Study)

| | Change from Baseline ITT Population | | |
|---|---|---|---|
| | (Median FEV1 74.75% at Baseline) Table | | |
| | Secondary Efficacy Endpoints in the ITT | | |
| | Population when R411 is Given as | | |
| | Monotherapy (ARES Study) | | |
| | Placebo | 200 mg | 600 mg |
| | (N = 117)/ | (N = 117)/ | (N = 119)/ |
| | [Mean BV] | [Mean BV]) | [Mean BV] |
| Rescue (2-agonist | 0.1/[2.98] | −0.36/[3.04] | −0.41*/[3.11] |
| use (puffs/d) | | | |
| Nocturnal | 0.06/[0.54] | −0.15*/[0.61] | −0.12*/[0.58] |
| awakenings | | | |
| (scores) | | | |
| Morning asthma | −0.13 [1.61] | −0.34*[1.55] | −0.33*[1.57] |
| symptoms | | | |
| Asthma control | −0.07 [2.00] | −0.29 [2.08] | −0.23 [2.00] |
| Questionnaire | | | |
| (Total Score) | | | |
| % Asthma | 32.50 | 25.6 | 26.10 |
| exacerbation | | | |

*p < 0.05, before adjustments for multiple comparisons; BV = baseline value for the group.

Solid Oral Dosage Forms Comprising Roflumilast and R411

In accordance with the present invention, solid pharmaceutical dosage forms for oral administration are provided comprising a therapeutically active amount of roflumilast (either as a racemic mixture or as a pure enantiomer), or a pharmaceutically acceptable salt thereof, a therapeutically effective amount of R411, or a pharmaceutically acceptable salt thereof, and one or more pharmaceutically acceptable excipients.

Without intending to limit the invention to any particular theory, the pharmaceutical dosage forms of the present invention are believed to provide an improved efficacy profile in the treatment of asthma by virtue of their complementary mechanisms of action. Roflumilast is a selective phosphodiesterase type-4 inhibitor, which is a bronchial dilator and exhibits respiratory drive increasing action and anti-inflammatory action. The specific mechanism of action of R411 suggests that it's greatest effect will be on the late-phase allergic response in animal and human challenge studies characterized by its effect on eosinophils. R411 inhibits eosinophil excitation and survival, inhibits eosinophil migration from blood to pulmonary tissues, and may promote apoptosis of tissue eosinophils though integrin blockade. Administration of a solid oral dosage form containing both roflumilast and R411 would therefore provide a therapeutic treatment having the combined effects of bronchial dilation and respiratory drive increasing action and reduction of eosinophil egress into pulmonary tissues. Hence administration of a dosage form containing both compounds provides a greater anti-inflammatory effect than that achieved by administration of either drug alone by virtue of their complementary modes of action.

The therapeutically effective amount or dosage of roflumilast and R411 according to this invention can vary within wide limits and may be determined in a manner known in the art. Such dosage will be adjusted to the individual requirements in each particular case including the specific compound(s) being administered, the condition being treated, as well as the patient being treated. In general, in the case of oral administration of roflumilast, or pharmaceutically acceptable salts thereof, to adult humans weighing approximately 70 Kg, roflumilast will be present in a daily dosage ranging from about 0.25 mg to about 0.50 mg. In general, in the case of oral administration of R411, or pharmaceutically acceptable salts thereof, to adult humans weighing approximately 70 Kg, R411 will be present in a daily dosage ranging from about 50 mg to about 400 mg, more preferably from about 50 mg to about 200 mg.

As set out above, because of the structural similarity of roflumilast and R411, it is possible to prepare a fixed dose combination product by admixing the two actives with pharmaceutically acceptable excipients. However, it is preferable that the two active ingredients be first pre-formulated separately to obtain pharmaceutically acceptable stability and bioavailability characteristics for each ingredient. The two separately pre-formulated active ingredients may then be combined in an appropriate solid dosage composition for oral administration. Particularly preferred solid dosage forms are those in which the separately pre-formulated ingredients are combined in a dosage form having separate discrete regions for the two pre-formulated ingredients such as by discrete layers, encapsulations, and the like. Examples of such dosage forms include, but are not limited to, a compressed tablet, a bilayer tablet, a sandwich tablet, a tablet having coated microbeads or film-coated tablets. These tablets could also designed to have fast disintegration in the oral cavity for administration to patients with swallowing difficulty such as children and the elderly.

In general, bilayer tablets may be formulated by utilizing twin hopper compression machines. The granulates of each compound may be prepared individually using pharmaceutically acceptable excipients such as lactose, sucrose, microcrystalline cellulose, stearic acid, hydroxypropylmethylcellulose, polyvinylpyrrolidone, crospovidone, croscarmelose sodium, sodium starch glycolate, dicalcium phosphate, mannitol, sorbitol, silicified microcrystalline cellulose, talc, colloidal silica, stearic acid, or magnesium stearate. The individual granulates can then be compressed together into one unit.

In general, sandwich tablets (or tablets inside tablets) can be prepared by sandwiching a tablet of roflumilast unit into the granulates of R411 using twin hopper compression machines. The tablet of roflumilast is prepared by using standard excipients described above and the granulates of R411 are prepared by conventional granulation techniques using pharmaceutically acceptable excipients.

In general, tablets having coated microbeads can be prepared by formulating one of the components, such as roflumilast, using either granulation or granulation followed by extrusion-merumerization techniques and coating the component with pharmaceutically acceptable polymers such as hypromellose, ethylcellulose, hydroxypropylcellulose, polyvinylalcohol, and/or aminomethylmethacrylate in fluid bed or coating pans in such a proportion that coating provides enough barrier to separate the two active components but does not affect the dissolution behavior of the coated product. The coated microbeads of roflumilast can then be mixed with R411 granulates prepared using conventional methods. These mixed granulations can be used to prepare tablets, capsules, or suspensions, or can be dispersed in an oily matrix. Separating the granulation process and further coating of those granulates help provide the barrier required to keep the two components separate while not affecting the dissolution behavior thus assuring the desired pharmacokinetic exposures. This approach can be used for taste masking each active separately and then mixing together with pharmaceutically acceptable excipients such as Pharmaburst® (SPI Pharma, Del.), crospovidone, FM1000 (J. M. Huber Corp, Md.), microcrystalline cellulose, colloidal silicone dioxide to yield an orally disintegrating product.

In general, film-coated tablets can be prepared by incorporating roflumilast in a film-coating layer. Tablets of R411 are prepared by conventional manufacturing processes such as granulation, milling, blending, lubricating, and compressing. The required dose of roflumilast is dissolved in a coating dispersion usually consisting of film forming agents such as hypromellose (hydroxypropyl methylcellulose), polyvinyl alcohol, starch or ethylcellulose along with a gliding agent such as talc, colorant and plasticizer (triacetin, dibutylsebacate, polyethylene glycol) dispersed in water. The required amount of roflumilast film coating is then applied over the R411 kernel tablet either in a pan coater or fluidbed coater to deposit the specific amount of roflumilast onto the R411 kernels.

The process of granulation consists of granulation with water or an appropriate solvent in a low or high shear granulator, fluid bed dryer, dry granulation with roller compaction or slugging or melt granulation using polyethylene glycols, phospholipids, poloxamers, monoglycerides, diglycerides and triglycerides, fatty acids, polyglycolized ester such as Gelucires, Vitamin E TPGS or by melt extrusion using thermosetting polymers such as polyvinylpyrrolidone, poloxamers, polyethylene glycol, ethyl cellulose, stearic acid, glyceryl monostearate, glyceryl behenate, and/or sucrose diesters. In order to manufacture the oral suspension, transdermal patches, these granulates in the desired proportion are dispersed in pharmaceutical bases consisting of excipients such as polyethylene glycols, surfactants Cremophor EL, Cremophor RH40, Solutol HS15, Gelucires 44/14, 50/15, 39/01, 33/01, polysorbates, spans, sodium dodecyl sulfate can be added to further improve the absorption process.

In another embodiment, the present invention provides a method for treating asthma comprising administering to a subject, in need thereof, a solid pharmaceutical dosage form for oral administration comprising a therapeutically active amount of roflumilast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients. Preferably, the dosage form comprises a combination of two discrete pre-formulated pharmaceutical compositions, the first composition comprising roflumilast and the second composition comprising R411. More preferably, the dosage form comprises two discrete regions, the first region comprising roflumilast and the second region comprising R411.

In yet another embodiment, the present invention provides a method for preparing a solid pharmaceutical dosage form for oral administration comprising admixing a therapeutically active amount of roflumilast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

The pharmaceutical compositions of the present invention may typically contain a fixed dose of from about 0. 1 mg to about 0.5mg of roflumilast or its corresponding salt or solvate form (e.g. hydrate) and from about 25 mg to about 400 mg of R411.

Roflumilast's incorporation into the combination dosage form of the present invention requires means to uniformly distribute the compound in a drug product, specifically in combination with R411, in addition to assuring the stability of the final dosage form. The primary degradation pathway is oxidation. Similarly, R411 being a weak base and possessing an ester linkage presents its challenges with regards to the hydrolytic stability.

The combination products of the two active components are amenable to being formulated in any finished dosage forms such as tablets, pills, capsules, powders, granules, films, wafers, oral solution, suspensions, drops for oral use and transdermal patches for administration via skin or suppositories for rectal administration. The preparation of fixed dose combinations can be manufactured by a variety techniques including any of the following methods:

1. Admixing the two actives (or pharmaceutically acceptable salt and solvate thereof) with pharmaceutically acceptable vehicles and then subdividing into unit dosage form (capsule, tablets or any suitable dosage form illustrated above).

2. The two actives are pre-formulated individually to yield two compositions such that roflumilast active formulated either as granules or tablets or microbeads or microtablets or microcapsules is combined with R411 active formulated as granules, tablets, microbeads or microtablets or micrcapsules to produce a unit dose product.

3. The two actives pre-formulated, as discussed above, are further coated to provide either a barrier coat for protection against degradation or to obtain modified release profile such as enteric-coating or to sustain the release or to mask the taste are mixed together to achieve a product with special attributes. The coatings can be designed with appropriate polymeric systems that provide different functionality.

The various approaches to these pharmaceutical compositions are exemplified below.

### EXAMPLES

A. Admixtures of Roflumilast and R411

The therapeutic quanitities of two actives are mixed together with pharmaceutically acceptable excipients and formulated into a unit dose such as tablets or capsules or dispersions using commonly used pharmaceutical operations. The selection of excipients and processes has to be optimized to ensure the manufacturability and stability of the product. Some of the excipients include but not limited to are lactose, sucrose, microcrystalline cellulose, stearic acid, hydroxypropylmethylcellulose, polyvinylpyrrolidone, crospovidone, croscarmelose sodium, sodium starch glycolate, dicalcium phosphate, corn starch, mannitol, sorbitol, xylitol, lactitol, maltitol, silcified microcrystalline cellulose, talc, colloidal silica, stearic acid, magnesium stearate sodium stearyl fumarate. Other excipients can be added to further improve the stability such as antioxidants (ascorbic acid, ascorbyl palmitate, sodium metabisulfite, butylhydroxytoluene or butylhydroxyanisole in appropriate amounts), pH-modifying agents such as citric acid, fumaric acid, tartaric acid, succinic acid, malic acid, adipic acid, arginine or lysine etc. An example illustrating such composition is shown below.

| | | |
|---|---|---|
| Roflumilast | 0.25 | mg |
| R411 | 100.00 | mg |
| Povidone K30 | 6.00 | mg |
| Crospovidone | 12.00 | mg |
| Starch 1500 | 10.00 | mg |
| Microcrystalline cellulose | 49.75 | mg |
| Ascorbic acid | 20.00 | mg |
| Lactose monohydrate | 150.00 | mg |
| Stearic acid | 6.00 | mg |

Manufacturing Procedure

1. Preparation of Ordered Mixtures: Ordered mixtures are frequently employed in pharmaceutical system to ensure content uniformity of the low dose active. The required weight of roflumilast is mixed with required amount of selected pharmaceutical excipients such as Starch 1500 (Colorcon, Pa.) or other equivalent materials such as corn starch or PureDent Starch (Grain Processing Corp, IL) in a appropriate blender (such as Turbula or PK blender with intensifier bar). The blend is then passed through 1 mm screen.

2. Granulation: The weighed quantity of lactose monohydrate is placed in a high shear granulator. The weighed quantity of R411 is placed on top followed by povidone K30, crospovidone ascorbic acid and roflumilast ordered mixture from Step 1. The required amount of granulating liquid (water, alcohol or combination thereof) is sprayed while continually mixing the contents until the desired granulation end point is reached. The granules are then screened through a course screen (3 mm). These granules are then dried either in tray ovens or in fluid bed dryers to the desirable moisture content (preferably less than 2% when determined at 90C using loss on drying apparatus). These granules are further milled to obtain granules of uniform size to allow for proper flow and compression. Finally the granules are mixed with external excipients such as microcrystalline cellulose and stearic acid in appropriate blender (such as bin blender).

The granules can also be prepared by alternate methods such as roller compaction, slugging, hot melt granulation using alternate binders such as polyethylene glycol 8000, polyglycolized fatty esters, poloxamers, etc., or hot melt extrusion using thermoplastic polymers, or high shear or spray granulation (using roflumilast preferably in the binder solution).

3. Compression: The granules prepared in Step 2 can be used in many different ways. If the desired product is tablet, the granules are compressed into tablet using appropriate tablet compression machines equipped with suitable tablet toolings (punches and dies) to the desired hardness.

4. Film-coating: Film-coating of compressed tablet is optional but is frequently used to enhance the elegance and ease the swallow-ability of the product. The tablets manufactured in Step 3 can be coated with standard film-coating material available for pharmaceutical use such as hypromellose with opacifying agents (talc), suitable colorants and plasticizers. Some of the preformulated systems available for this application are Opadry™ (Colorcon, Pa.), Chromatone™ (CHR Hansen, NJ). The film-coating is generally performed in perforated coating pans.

B. Pre-formulated Granules Combined Together to Obtain A Unit Dosage Form

The granulates of each components prepared individually using pharmaceutically acceptable excipients such as lactose, sucrose, microcrystalline cellulose, stearic acid, hydroxypropylmethylcellulose, polyvinylpyrrolidone, crospovidone, croscarmelose sodium, sodium starch glycolate, dicalcium phosphate, mannitol, sorbitol, silicified microcrystalline cellulose, talc, colloidal silica, stearic acid, magnesium stearate can be compressed together into one unit. The following example is illustrative (all percentages by weight unless otherwise indicated):

### Example 2

Roflumilast Granulation

| | |
|---|---|
| Roflumilast | 0.31% |
| Hypromellose | 5.00% |
| Crospovidone | 5.00% |
| Corn starch | 25.00% |
| Microcrystalline cellulose | 24.69% |
| Ascorbic acid | 6.25% |
| Lactose monohydrate | 32.50% |
| Stearic acid | 1.25% |

Granulation can be prepared by any of the method discussed in Example 1 provided that mixing of roflumilast is performed either by ordered mixing or by dispersing in the binder solution.

R411 Granulation:

| | |
|---|---|
| R411 | 50% |
| Povidone K30 | 4% |
| Crospovidone | 4% |
| Lactose monohydrate | 26% |
| Microcrystalline cellulose | 10% |
| Talc | 5% |
| Magnesium stearate | 1% |

Granulation can be prepared either by dry compression methods such as roller compaction, by high shear granulation or spray granulation using water or hydroalcohlic mixture as granulating fluid or by hot melt granulation (with a meltable binder) or hot melt extrusion (with thermoplastic polymers).

The unit dose product can be prepared in any of the following ways:

Compressed Tablets:

The required amount of each granulate is mixed together and compressed as a single unit. A tablet weighing 480 mg with 80 mg roflumilast granules and 400 mg R411 granules delivers 0.25 mg roflumilast and 200 mg R411.

Bilayer Tablets

The individually prepared granules of R411 and roflumilast are compressed together into a bilayer tablet using appropriate compression machines. A tablet containg 80 mg of roflumilast granules and 200 mg of R411 granules will yield 0.25 mg dose for roflumilast and 100 mg for R411.

Sandwich tablets

The roflumilast granules can be compressed into mini-tablets (0.25 mg per 80 mg). These tablets can then be placed inside R411 tablets during compression of R411 granules using specially equipped tableting machines.

Hard Gelatin Capsules

The granules prepared above can be filled into hard-gelatin capsules in an appropriate amount to obtain a unit dose formulation.

C. Roflumilast Microbeads

Roflumilast microbeads can be prepared either by extrusion-spheronization or by layering on inert sugar spheres or microcrystalline cellulose spheres. These microbeads can then be admixed with the R411 granules to be either compressed or filled into hard gelatin capsules. These microbeads can be further coated to obtain modified release profiles or to mask the unpleasant taste of the medicament or to provide a barrier to isolate the two components using appropriate polymers known to the person skilled the art. The commonly used excipients for barrier coating with sustained release applications include ethyllcellulose, copolymers of acrylates and methacrylates with quaternary ammonium (such as Eudragit RL, RS supplied by Rohm Pharma, NJ))The coating could be applied to the beads or granules using fluid bed apparatus. The following example illustrates such a composition manufactured by layering on non-pareil seeds.

### Example 3

| | |
|---|---|
| Roflumilast | 0.25% |
| Hypromellose | 10.00% |
| Ethylcellulose dispersion (Aquacoat ECD30) | 2.00% |
| Talc | 2.00% |
| Red iron oxide | 0.10% |
| Sugar spheres | 85.65% |

Manufacturing Procedure

The weighed amount of hypromellose, talc and pigment are dispersed in water using high shear homogenizer. Once a uniform dispersion is obtained the roflumilast, ethylcellulose dispersion are added to the dispersion and mixed gently using propeller mixer. This dispersion is then coated on to sugar spheres (60/80 mesh cut) in a fluid bed process with Wurster attachment. The microbeads obtained as above are mixed with R411 granules to produce either tablet or capsules.

Typical Manufacturing Procedure

A typical manufacturing procedure for tablets having coated microbeads is set out below. 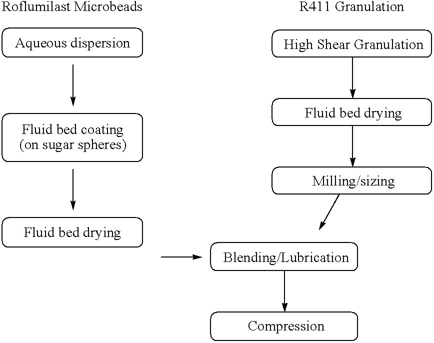
D. Film-Coating with Roflumilast

The R411 granules are manufactured as shown in Example 2. The granules are then compressed into tablets. The tablets are coated with aqueous dispersion of roflumilast along with other film-forming agents such as hypromellose. Other excipients such as talc, pigments (iron oxides or other colorants), plasticizers (triacetin, polyethylene glycol 8000) and opacifying agents (titanium dioxide) can be added to further enhance the film properties.

### Example 4

| | |
|---|---|
| R411 | 50.00% |
| Povidone K30 | 4.00% |
| Crospovidone | 4.00% |
| Lactose hydrous | 21.95% |
| Microcrystalline cellulose | 10.00% |
| Talc | 5.00% |
| Magnesium stearate | 1.00% |
| Roflumilast Film-Coat: | |
| Roflumilast | 0.05% |
| Coating Dispersion | 4.00% |

In this example, R411 granulation is compressed into a tablet to contain 200 mg R411. The compressed tablets are then film-coated with coating dispersion containing roflumilast A 400 mg film-coated tablet as shown in this example delivers 200 mg of R411 and 0.2 mg of roflumilast. The film-coat may comprise of any other film-forming polymer such as povidone-VA copolymer, ethycellulose, polyvinyl acetate, polyvinyl alcohol, polymethylmethacrylates with or without plasticizers (triacetin, triethyl citrate, dibutylsebacate, polyethylene glycol) etc. And the coating system can be dispersed in aqueous or non-aqueous media. The aqueous media may be appropriately buffered to achieve maximum solubility.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A solid pharmaceutical dosage form for oral administration comprising a therapeutically active amount of roflumilast, or a pharmaceutically acceptable salt thereof, a therapeutically effective amount of N-(2-chloro-6-methylbenzoyl)4-[(2,6-dichlorobenzoyl)amino]-L-phenylalanine-2-(diethylamino)ethyl ester, or a pharmaceutically acceptable salt thereof, and one or more pharmaceutically acceptable excipients.

2. The dosage form according to claim 1, wherein roflumilast is present in an amount from about 0.25 mg to about 0.5 mg.

3. The dosage form according to claim 1, wherein N-(2-chloro-6-methylbenzoyl)-4-[(2,6-dichlorobenzoyl)amino]-L-phenylalanine-2-(diethylamino)ethyl ester is present in an amount from about 50 mg to about 400 mg.

4. The dosage form according to claim 1, wherein the dosage form is selected from the group consisting of a compressed tablet, a bilayer tablet, a sandwich tablet, a tablet having coated microbeads, and a film coated tablet.

5. The dosage form according to claim 6, wherein the tablet having coated microbeads comprises:
(a) a tablet comprising N-(2-chloro-6-methylbenzoyl)-4-[(2,6-dichlorobenzoyl)amino]-L-phenylalanine-2-(diethylamino)ethyl ester present in an amount from about 50 mg to about 400 mg; and
(b) coated microbeads dispersed throughout the tablet comprising roflumilast present in an amount from about 0.25 mg to about 0.5 mg.

6. The dosage form according to claim 6, wherein the film coated tablet comprises:
(a) a tablet comprising N-(2-chloro-6-methylbenzoyl)-4-[(2,6-dichlorobenzoyl)amino]-L-phenylalanine-2-(diethylamino)ethyl ester present in an amount from about 50 mg to about 400 mg; and
(b) a film coat the tablet surface comprising roflumilast present in an amount from about 0.25 mg to about 0.5 mg.

7. The dosage form according to claim 1, comprising a combination of two pre-formulated pharmaceutical compositions, wherein a first composition of the two compositions comprises a therapeutically active amount of roflumilast, or a pharmaceutically acceptable salt thereof, which first composition is formulated with one or more pharmaceutically acceptable excipients; and a second composition of the two compositions comprises a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, which second composition is formulated with one or more pharmaceutically acceptable excipients.

8. The dosage form according to claim 7, wherein the two pre-formulated compositions are combined as two discrete regions in a single dosage form.

9. A method for treating asthma comprising administering to a subject, in need thereof, a solid pharmaceutical dosage form for oral administration comprising a therapeutically active amount of roflumilast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

10. The method according to claim 9, wherein roflumilast is present in an amount from about 0.25 mg to about 0.5 mg and N-(2-chloro-6-methylbenzoyl)-4-[(2,6-dichlorobenzoyl)amino]-L-phenylalanine-2-(diethylamino)ethyl ester is present in an amount from about 50 mg to about 400 mg.

11. The method according to claim 9, wherein the dosage form is selected from the group consisting of a compressed tablet, a bilayer tablet, a sandwich tablet, a tablet having coated microbeads, and a film coated tablet.
